(19) United States
(12) Patent Application Publication
(10) Pub. No.: US 2023/0078820 A1
(43) Pub. Date: Mar. 16, 2023

(54) FENFLURAMINE FOR TREATMENT OF DEMYELINATING DISEASES AND CONDITIONS

(71) Applicant: ZOGENIX INTERNATIONAL LIMITED, Berkshire (GB)

(72) Inventors: John Chang-Eun CHA, San Mateo, CA (US); Thaddeus Cromwell REEDER, San Carlos, CA (US)

(73) Assignee: ZOGENIX INTERNATIONAL LIMITED, Berkshire (GB)

(21) Appl. No.: 17/895,443

(22) Filed: Aug. 25, 2022

Related U.S. Application Data
(60) Provisional application No. 63/239,801, filed on Sep. 1, 2021.

(51) Int. Cl. A61K 31/137; A61P 25/28

(52) U.S. Cl. CPC A61K 31/137; A61P 25/28

(57) ABSTRACT

Methods of modifying disease progression of a neurodegenerative disease or condition associated with demyelination, insufficient myelination, or underdevelopment of myelin sheath are also described herein.

### FIELD OF THE INVENTION

The invention relates to the treatment of diseases or conditions associated with demyelination owing to various pathologic mechanisms, insufficient myelination, or underdevelopment of the myelin sheath. More specifically, the invention relates to the use of fenfluramine for the treatment of such diseases and conditions.

### BACKGROUND

Oligodendrocytes (OL) generate and maintain myelin in the central nervous system (CNS). During development, oligodendrocyte precursor cells (OPC) differentiate into OL. However, damaged or injured white matter frequently does not remyelinate and currently available treatments of demyelinating diseases are limited in their efficacy. For some demyelinating diseases, there are no known treatments available. Thus, therapeutic agents capable of promoting remyelination or slowing or halting demyelination represent an unmet medical need.

### SUMMARY

The present disclosure features the use of a CNS active drug, fenfluramine, or a pharmaceutically acceptable salt thereof in an amount capable of reducing demyelination and promoting myelination. Fenfluramine, and pharmaceutically acceptable salts thereof, provide a viable treatment for preventing and reversing demyelination in disorders such as multiple sclerosis (MS), epileptic encephalopathies and other diseases or conditions associated with demyelination, insufficient myelination, or underdevelopment of the myelin sheath. Fenfluramine is an amphetamine derivative drug that was once widely prescribed as an appetite suppressant to treat obesity. Fenfluramine is devoid of the psychomotor stimulant and abuse potential of D-amphetamine and interacts with certain 5-hydroxytryptamine (serotonin, 5-HT) receptors and the serotonin transporter to release 5-HT from neurons. Low dose fenfluramine has been shown to provide anticonvulsive activity in the treatment of Dravet Syndrome, previously known as severe myoclonic epilepsy in infancy or SMEI, and Lennox Gastaut syndrome, both rare and malignant epileptic syndromes. Fenfluramine is approved for use in the treatment of Dravet syndrome under the brand name Fintepla® in the United States and parts of the European Union. About four decades before its reintroduction, fenfluramine was approved for use in treatment of obesity, and often combined with phentermine for that purpose, the combination that became a cultural phenomenon dubbed fen-phen. Fenfluramine was removed from the market worldwide when it was found that in some patients it induced cardiac valve abnormalities or pulmonary hypertension.

Fenfluramine is also referred to as 3-trifluoromethyl-N-ethylamphetamine and has the structure:

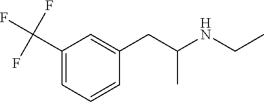 [SMILES: CCNC(C)Cc1cccc(C(F)(F)F)c1]

Systematic nomenclature for racemic fenfluramine is (RS)-N-ethyl-1-[3-(trifluoromethyl) phenyl]propan-2-amine

The inventors have discovered that fenfluramine interacts with RNF43.

RING finger protein 43 (RNF-43) is a membrane bound receptor that down regulates Wnt signaling. There are multiple signaling pathways for Wnt signaling, of which the three best characterized signaling pathways are: the canonical Wnt pathway; the noncanonical planar cell polarity pathway; and the noncanonical Wnt/calcium pathway. The difference between the categories is that a canonical pathway involves the protein β-catenin while the noncanonical pathways operate independent of it. The Wnt-β-catenin signaling pathway is tightly controlled at multiple levels by ubiquitination, and dysregulation of these pathways promotes tumorigenesis. RNF43 is a ubiquitin ligase originally found in stem cells and proposed to inhibit Wnt signaling by interacting with the Wnt receptors of the Frizzled family [Loregger A, et al., Sci Signal. 2015 Sep. 8; 8(393):ra90. doi: 10.1126/scisignal.aac6757]. Mutations of RNF43 are observed in many cancers, particularly colon malignancies and are driven by high Wnt signaling tone. Functional RNF43 suppresses both Wnt/β-catenin signaling and noncanonical Wnt signaling by distinct mechanisms. The suppression of Wnt/β-catenin signaling requires interaction between the extracellular protease-associated (PA) domain and the cysteine-rich domain (CRD) of frizzled and the intracellular RING finger domain (N-terminal domains) of RNF43. In contrast, these N-terminal domains of RNF43 are not required for inhibition of noncanonical Wnt signaling but rather an interaction between the C-terminal cytoplasmic region of RNF43 and the PDZ domain of dishevelled is essential for this type of suppression.

Myelin is an insulating layer, or sheath that forms around the axons of nerve cells, including both the brain and spinal cord, and is made up of protein and fatty substances. The process of creating myelin is performed by oligodendrocytes. The myelin sheath allows electrical impulses to transmit quickly and efficiently along the nerve cells. If myelin is damaged, these impulses slow down or are weakened. Oligodendrocytes are the cellular casualties of injury by a large variety of mechanisms including, but not limited to, autoimmune injury associated with multiple sclerosis and neonatal white matter injuries from hypoxia, developmental disorders or disease which can result in cerebral palsy. Work from Stephen Fancy's lab (UCSF) reported parallel states of pathological Wnt signaling in neonatal brain injury and colon cancer and demonstrated that RNF-43 is not required for normal myelination but is required for myelin repair after white matter damage (hypoxia). [Fancy, S P J et al., Nat Neurosci. 2014 April; 17(4): 506-512]

Following hypoxic-ischemic injury to white matter oligodendrocyte precursor cells (OPCs), it has been demonstrated that β-catenin-mediated TCF (T cell factor) dependent transcription increased, while expression of its negative regulator adenomatous polyposis coli (APC) protein fell. The injured cells mimic the dysregulated Wnt/βcatenin-signaling noted in APC-deficient colon cancers, in that they share up-regulation of a discrete set of Wnt-modulated transcriptional target genes. The tonically upregulated, “feed-forward” nature of dysregulated over-expression of these genes appears to render OPCs refractory to terminal oligodendrocyte differentiation, instead favoring their persistence in the progenitor state. This mechanism results in suppression of remyelination.

Unlike the neoplastic expansion seen in APC-mutant or deficient colon carcinomas, the sustained Wnt/βcatenin signal activation and diminished APC expression seen in OPCs following injury is associated with maturational arrest of OPCs, and not with facilitated cell division or tumorigenesis, thus highlighting the effects of phenotype and local tissue context on the cellular outcomes of dysregulated Wnt signaling. [Goldman, S. et al., Nat Neurosci. 2014 April; 17(4): 483-485].

The present inventors have demonstrated that fenfluramine reduces myelin damage using antibody staining techniques to show a reduction of damaged myelin basic protein and fewer CD11b+ microglia (CNS parenchymal macrophages), indicators of myelin damage and removal respectively. Fenfluramine binds to RNF43 and may enhance RNF43's ability to down regulate the effects of high Wnt signaling on OPCs, especially those in damaged/demyelinated white matter. Their studies were conducted in treated and untreated mice having a SCN1A gene mutation, which produces a murine model of Dravet syndrome. With modern neuro-imaging techniques, it has been shown that various epilepsies exhibit white matter abnormalities that may be a part of the epileptogenic process in developing recurring seizures. [Hatton S N, Huynh K H, Bonilha L, et al. Brain. 2020; 143(8):2454-2473] In addition to administering fenfluramine to study animals, the inventors dosed other members of the Dravet mice cohort with diazepam, a benzodiazepine known to be effective in acute seizure reduction. The data show that Diazepam does not appear therapeutically useful in reducing myelin debris or CD11b+ microglia. Without being bound by a particular theory, it is believed that fenfluramine binding to RNF-43 provides for a specific, myelin-repairing activity of fenfluramine in demyelination and hypomyelination related disease states.

Methods of treating a subject having or at risk of developing a neurodegenerative disease or condition associated with demyelination, insufficient myelination, or underdevelopment of myelin sheath are described herein.

Methods of modifying disease progression of a neurodegenerative disease or condition associated with demyelination, insufficient myelination, or underdevelopment of myelin sheath are also described herein.

The methods include administration of a therapeutically effective amount of fenfluramine, or a pharmaceutically acceptable salt thereof.

A method of the invention includes a treatment, comprising:

administering to a subject diagnosed with a demyelination disorder a therapeutically effective amount of fenfluramine or a pharmaceutically acceptable salt thereof.

A method of the invention includes a treatment comprising:

the demyelination disorder is multiple sclerosis (MS).

Another method of the invention includes a treatment wherein the demyelination disorder is an epileptic encephalopathy.

Another method of the invention includes a treatment wherein the demyelination disorder is insufficient myelination.

Another method of the invention includes a treatment wherein the demyelination disorder is underdevelopment of myelin sheaths.

A method of the invention includes a treatment comprising:

diagnosing a patient with a condition associated with demyelination.

administering to the patient a therapeutically effective amount of fenfluramine or a pharmaceutically acceptable salt thereof.

Another method of the invention includes a treatment wherein the condition associated with demyelination is selected from the group consisting of multiple sclerosis, a leukodystrophy, a leukoencephalopathy, an idiopathic inflammatory demyelinating disease, and Alzheimer's disease.

Another method of the invention includes a treatment wherein the condition associated with demyelination is multiple sclerosis of a type selected from the group consisting of relapsing-remitting multiple sclerosis, primary-progressive multiple sclerosis, secondary-progressive multiple sclerosis, and progressive-relapsing multiple sclerosis.

Another method of the invention includes a treatment wherein the condition associated with demyelination is selected from the group consisting of central pontine myelinolysis, acute disseminated encephalomyelitis, Balo concentric sclerosis, Marburg multiple sclerosis, tumefactive multiple sclerosis, diffuse myelinoclastic sclerosis, acute hemorrhagic leukoencephalitis, neuromyelitis optica, a chronic inflammatory demyelinating polyneuropathy, Leber hereditary optic neuropathy, multifocal motor neuropathy, paraproteinemic demyelinating polyneuropathy, tropical spastic paraparesis, a Guillain-Barre syndrome, infantile Refsum disease, adult Refsum disease 1, adult Refsum disease 2, Zellweger syndrome, X-linked 5adrenoleukodystrophy (X-ALD), metachromatic leukodystrophy, Krabbe disease, Pelizaeus-Merzbacher disease, Canavan disease, Alexander disease, Binswanger's disease, peroneal muscular atrophy, cerebrotendineous xanthomatosis, leukoencephalopathy with vanishing white matter, toxic leukoencephalopathy, van der Knaap disease, progressive multifocal leukoencephalopathy, Marchiafava-Bignami disease, and transverse myelitis.

A method of the invention includes a treatment comprising:

diagnosing a patient with a symptom of a disease;

administering to the patient a therapeutically effective amount of fenfluramine or a pharmaceutically acceptable salt thereof,

wherein the symptom is selected from the group consisting of a lack of sphincter control, erectile dysfunction, paraparesis, ataxia, adrenocortical insufficiency, progressive neuropathy, paresthesia, dysarthria, dysphagia, clonus, or any combination thereof.

A method of the invention includes a treatment comprising administering via a route selected from the group consisting of orally, parenterally, and topically.

A method of the invention includes a treatment comprising:

wherein the fenfluramine or pharmaceutically acceptable salt thereof is administered at a dose of about 0.5 mg/kg/day to about 5 mg/kg/day.

A method of the invention includes a treatment comprising:

wherein the fenfluramine or pharmaceutically acceptable salt thereof is administered at a dose of about 0.1 mg/kg/day to about 2 mg/kg/day.

A method of the invention includes a treatment comprising:

wherein the administering is at an interval selected from the group.

“Methods of treatment” are referred to throughout this disclose and such includes any “use” of the composition, compound or formulation for the intended purpose or any “manufacture for use” for he intended purpose.

Any reference in the description to “methods of treatment” or “in vivo diagnosis” is to be interpreted as referring to compounds, pharmaceutical compositions and medications of the present invention for use in methods of treatment of the human or animal body by therapy or for in vivo diagnosis, and/or the manufacture of compounds, pharmaceutical compositions and medications of the present invention for use in methods of treatment of the human or animal body by therapy or for in vivo diagnosis.

The foregoing aspects, embodiments, and advantages of the invention will become more apparent from the following detailed description, which proceeds with reference to the accompanying figures.

### BRIEF DESCRIPTION OF THE DRAWINGS

FIG. 1 consists of four separate panels (fenfluramine applied at 1 micromolar, 5 micromolar concentrations, no fenfluramine control and a protein identity legend) and shows the binding interaction between fenfluramine and the RNF43 protein.

FIG. 2 is a box and whisker plot showing that fenfluramine significantly reduced the myelin debris (D-MBP) score in the hippocampus (hipp) and cortex (ctx) of P35-P37 SCN1a+/− mice (a model of Dravet Syndrome) in comparison to vehicle (saline placebo) treatment with a 2-tail, unequal variance t-test significance of p=0.01. Vehicle-treated, P35-P37 non-mutant (SCN1a+/+, wild-type) mice were compared to fenfluramine treated, vehicle treated (placebo), and diazepam treated P35-P37 SCN1a+/− (Dravet) mice for levels of D-MBP staining. Fenfluramine consistently reduced D-MBP staining scores compared to diazepam treated cohorts of the same SCN1a+/− heterozygous (Dravet) mice. This graph indicates that fenfluramine reduces levels of damaged myelin. D-MBP Average Score in Cortex and Hippocampus of P35-P37 SCN1A+/+(wild-type) and SCN1A+/− (Dravet) mice in each cohort indicated by n=number. **=Fenfluramine vs Saline (p=0.01). Diazepam vs Saline p=not significant). See also Example 2.

FIG. 3 consists of 3A, 3B and 3C where 3A is a bar graph showing differences in D-MBP binding and scoring between P37-P40 non-mutant (SCN1a+/+, wild-type) and P37-P40 SCN1a+/− heterozygous (Dravet) mice (see also Example 2). Dravet mice have increased degraded myelin debris compared to wildtype mice at P37-P40 with a 2-tail, equal variance t-test significance of p=0.003. The number of animals in each experimental cohort in indicated by n=number. This graph indicates that the mice that have the Dravet mutation (SCN1a+/−) have increased levels of damaged myelin. D-MBP average score in P37-P40 SCN1A+/+ (wild-type) and SCN1A+/− (Dravet) mice. The sum of the individual cortex and hippocampus scores are averaged to give D-MBP average score. P-value between the wild-type and Dravet group is p=0.003. 3B is a histology photo of myelin staining in P37-P40 SCN1a+/− (Dravet) mouse brain tissue and 3C is a histology photo of myelin staining in wildtype (WT) P37-P40 mouse brain tissue. Dark blue/black areas indicate areas of degraded myelin basic protein.

FIG. 4 is a box and whisker plot showing differences in grading counts of activated CD11b+ microglia in the corpus callosum and hippocampus from vehicle-treated, P35-P37 non-mutant (SCN1a+/+, wild-type) mice, and fenfluramine treated, vehicle (saline placebo) treated, and diazepam treated groups of P35-P37 SCN1a+/− (Dravet) mice. The plot shows the sum of the scoring of the genu of the corpus callosum and the perforant pathway (p.path.) of the hippocampus. Fenfluramine reduced activated microglia in the Dravet mice compared to vehicle (saline) with a 2-tail, unequal variance t-test significance of p=0.05. The effect of Diazepam on levels of CD11b+ microglia was not significant compared to the vehicle treated group. This graph indicates that fenfluramine reduces levels of the activated microglia involved in the removal of damaged myelin, consistent with the data showing that fenfluramine reduced levels of the D-MBP marker for damaged myelin. CD11b+ Activated Microglia Average Score in Genu and Perforant Pathway of P35-P37 SCN1A+/+ (wild-type) and SCN1A+/− (Dravet) mice. Number of mice in each cohort indicated by n=number). **=Fenfluramine vs Saline (p=0.05). Diazepam vs Saline p=not significant).

FIG. 5 is a box and whisker plot showing the differences in CD11b+ activated microglia in areas of the corpus callosum and hippocampus between P37-P40 wildtype and P37-P40 SCN1a+/− (Dravet) mice. Dravet mice have increased CD11b+ activated microglia compared to wildtype mice at P37-P40 with a 2-tail, unequal variance t-test significance of p=0.002. The number of animals in each experimental cohort in indicated by n=number. This graph supports the observation that the mice that have the Dravet mutation (SCN1a+/−) have increased levels of damaged myelin. CD11b average score in P37-P40 SCN1A+/+ (wild-type) and SCN1A+/− (Dravet) mice. The sum of the individual genu and perforant scores are averaged to give the CD11b average score. P-value between the wild-type and Dravet group is p=0.002.

FIG. 6 is a graph of a Kaplan Meier estimation showing that fenfluramine (FFA) treatment increases the probability of survival over time of P1-P40 in SCN1a+/− (het) mice compared to both placebo and diazepam treatment.

### DETAILED DESCRIPTION

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

I. Abbreviations

ADEM acute disseminated encephalomyelitis

AIDP acute inflammatory demyelinating polyneuropathy

CIDP chronic inflammatory demyelinating polyneuropathy

CNS central nervous system

DMBP (or D-MBP) degraded myelin basic protein

FFA fenfluramine hydrochloride

IIDD idiopathic inflammatory demyelinating disease

i.p. intraperitoneal

MOG myelin oligodendrocyte glycoprotein

MS multiple sclerosis

NMO neuromyelitis optica

OL oligodendrocytes

OPC oligodendrocyte precursor cells

PML progressive multifocal leukoencephalopathy

SCN1a+/− an organism having one null allele and one functional allele for the sodium channel 1 alpha subunit of the NaV1.1 sodium channel

T3 triiodothyronine

X-ALD X-linked adrenoleukodystrophy

II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Acute disseminated encephalomyelitis (ADEM): An immune-mediated demyelinating disease of the central nervous system. ADEM usually occurs following a viral infection, but may also appear following vaccination or following bacterial or parasitic infection. In some cases. ADEM develops spontaneously. The disease involves autoimmune demyelination, similar to multiple sclerosis, and is therefore considered a multiple sclerosis borderline disease. ADEM produces multiple inflammatory lesions in the brain and spinal cord, particularly in the white matter. The lesions are typically found in the subcortical and central white matter and cortical gray-white junction of both cerebral hemispheres, cerebellum, brainstem, and spinal cord, but periventricular white matter and gray matter of the cortex, thalami and basal ganglia may also be involved. When a patient suffers more than one demyelinating episode, the disease is referred to as recurrent disseminated encephalomyelitis or multiphasic disseminated encephalomyelitis.

Acute hemorrhagic leukoencephalitis (AHL or AHLE): A hyperacute and frequently fatal form of ADEM. This disease is also known as acute necrotizing encephalopathy (ANE), acute hemorrhagic encephalomyelitis (AHEM), acute necrotizing hemorrhagic leukoencephalitis (ANHLE), Weston-Hurst syndrome, or Hurst's disease.

Administration: To provide or give a subject an agent, such as a therapeutic agent (e.g. fenfluramine or a pharmaceutically acceptable salt thereof), by any effective route. Exemplary routes of administration are described hereinbelow.

Adult Refsum disease: An autosomal recessive neurological disease that is associated with the over-accumulation of phytanic acid in cells and tissues. Adult Refsum disease is divided into the adult Refsum disease 1 and adult Refsum disease 2 subtypes. Individuals with Refsum disease present with neurologic damage, cerebellar degeneration, and peripheral neuropathy. Onset is most commonly in childhood/adolescence with a progressive course, although periods of stagnation or remission occur. Symptoms also include ataxia, scaly skin (ichthyosis), difficulty hearing, and eye problems including cataracts and night blindness.

Alexander disease: A very rare, congenital demyelinating disease. The disease primarily affects infants and children, causing developmental delay and changes in physical characteristics. Alexander disease is a type of leukodystrophy.

Alzheimer's disease: The most common form of dementia. Symptoms of Alzheimer's disease include memory loss, confusion, irritability, aggression, mood swings and trouble with language. This disease is characterized by the loss of neurons and synapses in the cerebral cortex and certain subcortical regions. The loss results in gross atrophy of the affected regions, including degeneration in the temporal lobe, and parts of the frontal cortex and cingulate gyrus. Amyloid plaques and neurofibrillary tangles are visible by microscopy in brains of those afflicted with this disease. The cause of Alzheimer's disease is unknown; however, several hypotheses exist, including that the disease is caused by age-related myelin breakdown in the brain. [Bartzokis, G. Neurobiology of Aging Volume 25, Issue 1, January 2004, Pages 5-18]

Balo concentric sclerosis: A demyelinating disease similar to standard multiple sclerosis, but with the particularity that the demyelinated tissues form concentric layers. Patients with this disease can survive and/or have spontaneous remission. Typically, the clinical course is primary progressive, but a relapsing-remitting course has been reported.

Canavan disease: An autosomal recessive degenerative disorder that causes progressive damage to nerve cells in the brain. Canavan disease is a leukodystrophy and is one of the most common degenerative cerebral diseases of infancy. This disease is also called Canavan-Van Bogaert-Bertrand disease, aspartoacylase deficiency and aminoacylase 2 deficiency.

Central pontine myelinolysis (CPM): A neurologic disease caused by severe damage of the myelin sheath of nerve cells in the brainstem, more precisely in the area termed the pons. The most common cause is the rapid correction of low blood sodium levels (hyponatremia). Frequently observed symptoms in this disorder are sudden para or quadraparesis, dysphagia, dysarthria, diplopia and loss of consciousness. The patient may experience locked-in syndrome where cognitive function is intact, but all muscles are paralyzed with the exception of eye blinking.

Cerebral palsy: A term used for a group of permanent, non-progressive movement disorders that cause physical disability. Cerebral palsy is caused by damage to the motor control centers of the developing brain and can occur during pregnancy, during childbirth, or after birth up to about age three. Patients with cerebral palsy exhibit damage to myelin sheaths.

Cerebrotendinous xanthomatosis: An inherited disorder associated with the deposition of a form of cholesterol (cholestanol) in the brain and other tissues and with elevated levels of cholesterol in plasma but with normal total cholesterol level. It is characterized by progressive cerebellar ataxia beginning after puberty and by juvenile cataracts, juvenile or infantile onset chronic diarrhea, childhood neurological deficit, and tendinous or tuberous xanthomas. This disorder is an autosomal recessive form of xanthomatosis. It falls within a group of genetic disorders called the leukodystrophies.

Chronic inflammatory demyelinating polyneuropathy (CIDP): An acquired immune-mediated inflammatory disorder of the peripheral nervous system. The disorder is sometimes called chronic relapsing polyneuropathy (CRP) or chronic inflammatory demyelinating polyradiculoneuropathy (because it involves the nerve roots). CIDP is closely related to Guillain-Barre syndrome and it is considered the chronic counterpart of that acute disease. Its symptoms are also similar to progressive inflammatory neuropathy. An asymmetrical variant of CIDP is known as Lewis-Sumner syndrome. The pathologic hallmark of the disease is loss of the myelin sheath.

Demyelinating disease: Includes any disease of the nervous system in which myelin is damaged or lost, or in which the growth or development of the myelin sheath is impaired. Demyelination inhibits the conduction of signals in the affected nerves, causing impairment in sensation, movement, cognition, or other functions for which nerves are involved. Demyelinating diseases have a number of different causes and can be hereditary or acquired. In some cases, a demyelinating disease is caused by an infectious agent, an autoimmune response, a toxic agent or traumatic injury. In other cases, the cause of the demyelinating disease is unknown (“idiopathic”) or develops from a combination of factors.

Devic's syndrome: An autoimmune, inflammatory disorder in which a person's immune system attacks the optic nerves and spinal cord, which results in inflammation of the optic nerve (optic neuritis) and the spinal cord (myelitis). Spinal cord lesions lead to varying degrees of weakness or paralysis in the legs or arms, loss of sensation, and/or bladder and bowel dysfunction. Although inflammation may also affect the brain, the lesions are different from those observed in MS. Devic's disease is similar to MS in that the body's immune system attacks the myelin surrounding nerve cells. Unlike standard MS, the attacks are not believed to be mediated by the immune system's T cells but rather by antibodies called NMO-IgG. These antibodies target a protein called aquaporin 4 in the cell membranes of astrocytes which acts as a channel for the transport of water across the cell membrane. Devic's syndrome is also known as Devic's disease or neuromyelitis optica (NMO).

Diffuse myelinoclastic sclerosis: An uncommon neurodegenerative disease that presents clinically as pseudotumoral demyelinating lesions. It usually begins in childhood, affecting children between 5 and 14 years old; however, cases in adults are possible. This disease is considered one of the borderline forms of MS and is sometimes referred to as Schilder's disease.

Epileptic encephalopathies: This group of disorders includes Ohtahara syndrome, Early myoclonic encephalopathy, West syndrome, Dravet syndrome, Lennox-Gastaut syndrome, Epileptic encephalopathy with continuous spike-and-wave during sleep (CSWS), Landau-Kleffner syndrome (LKS) and other conditions are under evaluation for inclusion in this group, including CDKLS deficiency disorder, Rett syndrome, and Doose syndrome. The term “epileptic encephalopathies” refers to a group of disorders in which unremitting or refractory epileptic activity contributes to severe cognitive and behavioral impairments which goes beyond what might be expected from the underlying pathology alone and the encephalopathy can worsen over time leading to progressive cerebral dysfunction. [Jain, Puneet et al. Epilepsy research and treatment vol. 2013 (2013): 501981. doi:10.1155/2013/501981]. Some of these disorders are monogenetic in origin. For instance, Dravet syndrome is associated with mutations of the SCN1A gene in upwards of 80% of cases, Rett syndrome is associated with mutations in MECP2, and CDKL5 deficiency disorder, previously believed to be a variant of Rett, is associated with mutations in CDKL5. In other of these disorders the etiology is not well understood, such as, for example, Lennox Gastaut syndrome and West syndrome. Dravet patients have loss of white matter as demonstrated by imaging [Perez, A., et al. Epilepsy Res. 2014 October; 108(8):1326-34] and by histopathology of brain tissue from deceased patients. [Catarino, C., et al. Brain 2011. 134 2982-3010] Myelin pathology including hypomyelination and demyelination was seen in all resected epileptogenic lesions the inventors examined for myelin from human FCD, non-genetic epilepsy, OGD, and TSC patients. Demyelination is a both a consequence and cause of epileptic disorders.

Tuberous sclerosis complex (TSC) is caused mutations in TSC1 or TSC2 genes that encode for the proteins hamartin and tuberin respectively. The mutations affect numerous tissues; patients have heart, kidney, and skin lesions and neurological issues, which are most problematic. One of the diagnostic features of TSC are malformations called cortical tumors that arise from TSC is a neurodevelopmental disorder that results in developmental delay and intellectual disability. Ninety percent of patients have one or more TSC-associated neuropsychiatric disorders (TANDs) which include behavioral, psychiatric, neuropsychological, and social/emotional processing issues. A significant proportion of TSC patients (˜50%) are diagnosed with an autism spectrum disorder. An estimated 80% of patients have seizures and neuronal hyperexcitability, as measured by electroencephalogram (EEG), and spasms frequently appear in infancy, manifesting as small spasms characterized by coincident loss of truncal tone and a sudden increase in tonicity leading to a head-bobbing motion. Hyperexcitability also manifests as focal seizures in TSC patients. Experimentally, TSC1 gene deletions in radial glia and radial glia deleted progeny of mice causes mis-lamination, macrocephaly, cytomegaly, hypomyelination, and reactive gliosis with seizures and TSC2 deletion by hGFAP-CRE in mice also generates macrocephalic mice having cytomegaly, hypomyelination, reactive gliosis, and seizures.

Encephalomyelitis: Inflammation of the brain and spinal cord.

Guillain-Barre syndrome: An acute polyneuropathy, a disorder affecting the peripheral nervous system. Ascending paralysis, weakness beginning in the feet and hands and migrating towards the trunk, is the most typical symptom, and some subtypes cause change in sensation or pain, as well as dysfunction of the autonomic nervous system. It can cause life-threatening complications, in particular if the respiratory muscles are affected or if the autonomic nervous system is involved. This disease is usually triggered by an infection. Acute inflammatory demyelinating polyneuropathy (AIDP) is the most common subtype of this disease. Other subtypes of Guillain-Barre syndrome include Miller Fischer syndrome, acute motor axonal neuropathy (Chinese paralytic syndrome), acute motor sensory axonal neuropathy, acute panautonomic neuropathy, and Bickerstaffs brainstem encephalitis.

Hemorrhage: Bleeding or escape of blood from a vessel, including hemorrhagic stroke or burst aneurysms.

Hypoxia: The lack of oxygen supply to the tissues of the body below the normal level. Hypoxia can be caused by many different factors, which include drowning, smoke inhalation, strangulation, hemorrhagic or ischemic stroke, asthma, prolonged seizures epileptic seizures. Cerebral hypoxia refers to the brain not receiving or not being able to process enough oxygen. Premature infants often suffer cerebral hypoxia in the days and weeks after birth which is often associated with persistent motor (including cerebral palsy), sensory, and cognitive impairment. Idiopathic inflammatory demyelinating disease (IIDD): A broad spectrum of central nervous system disorders that can usually be differentiated on the basis of clinical, imaging, laboratory and pathological findings. Idiopathic inflammatory demyelinating diseases are sometimes known as borderline forms of multiple sclerosis. IIDD generally refers to a collection of multiple sclerosis variant diseases, including but not limited to, optic-spinal MS, Devic's disease, ADEM, acute hemorrhagic leukoencephalitis, Balo concentric sclerosis, Schilder disease, Marburg multiple sclerosis, tumefactive multiple sclerosis and solitary sclerosis.

Infantile Refsum disease: A peroxisome biogenesis disorder associated with deficiencies in the catabolism of very long chain fatty acids and branched chain fatty acids (such as phytanic acid) and plasmalogen biosynthesis. Infantile Refsum disease is a rare, autosomal recessive congenital disorder, and one of three peroxisome biogenesis disorders that belong to the Zellweger spectrum of peroxisome biogenesis disorders.

Injury: Refers to any type of physical damage to cells, tissues, or the body. In some cases, nervous system (e.g., CNS or PNS) injury results in demyelination and/or a demyelinating disease.

Ischemia: A vascular phenomenon in which a decrease in the blood supply to a bodily organ, tissue, or part is caused, for instance, by constriction or obstruction of one or more blood vessels. Ischemia sometimes results from vasoconstriction, thrombosis or embolism. Ischemia can lead to direct ischemic injury, tissue damage due to cell death caused by reduced oxygen supply. In some cases, ischemia can lead to demyelination.

Krabbe disease: A rare, often fatal degenerative disorder that affects the myelin sheath of the nervous system. It is a form of sphingolipidosis, as it involves dysfunctional metabolism of sphingolipids. This condition is inherited in an autosomal recessive pattern. Krabbe disease is also known as globoid cell leukodystrophy or galactosylceramide lipidosis.

Leber hereditary optic neuropathy: A mitochondrially inherited (transmitted from mother to offspring) degeneration of retinal ganglion cells (RGCs) and their axons that leads to an acute or subacute loss of central vision; this affects predominantly young adult males.

Leukodystrophy: Refers to a group of diseases that affects the growth or development of the myelin sheath.

Leukoencephalopathy: Any of a group of diseases affecting the white substance of the brain; can refer specifically to several diseases including, for example, “leukoencephalopathy with vanishing white matter” and “toxic leukoencephalopathy.” Leukoencephalopathies are leukodystrophy-like diseases.

Marburg multiple sclerosis: A condition in which the central nervous system has multiple demyelinating lesions with atypical characteristics for those of standard multiple sclerosis. This disease is a borderline form of multiple sclerosis and is also known as tumefactive multiple sclerosis or fulminant multiple sclerosis. It is called tumefactive because the lesions are “tumor-like” and they mimic tumors clinically, radiologically and sometimes pathologically.

Marchiafava-Bignami disease: A progressive neurological disease characterized by corpus callosum demyelination and necrosis and subsequent atrophy. It is classically associated with chronic alcoholics.

Metachromatic leukodystrophy (MLD): A lysosomal storage disease that is commonly listed in the family of leukodystrophies, as well as in the sphingolipidoses as it affects the metabolism of sphingolipids. MLD is directly caused by a deficiency of the enzyme arylsulfatase A.

Multifocal motor neuropathy (MMN): A progressively worsening condition where muscles in the extremities gradually weaken. This disorder, a motor neuropathy syndrome, is sometimes mistaken for amyotrophic lateral sclerosis (ALS) because of the similarity in the clinical picture, especially if muscle fasciculations are present. MMN is usually asymmetric and is thought to be autoimmune.

Multiple sclerosis (MS): A slowly progressive CNS disease characterized by disseminated patches of demyelination in the brain and spinal cord, resulting in multiple and varied neurological symptoms and signs, usually with remissions and exacerbation. The cause of MS is unknown, but an immunological abnormality is suspected. An increased family incidence suggests genetic susceptibility, and women are somewhat more often affected than men. The symptoms of MS include weakness, lack of coordination, paresthesia, speech disturbances, and visual disturbances, most commonly double vision. More specific signs and symptoms depend on the location of the lesions and the severity and destructiveness of the inflammatory and sclerotic processes. Relapsing-remitting multiple sclerosis (RRMS) is a clinical course of MS that is characterized by clearly defined, acute attacks with full or partial recovery and no disease progression between attacks. Secondary-progressive multiple sclerosis (SPMS) is a clinical course of MS that initially is relapsing-remitting, and then becomes progressive at a variable rate, possibly with an occasional relapse and minor remission. Primary-progressive multiple sclerosis (PPMS) presents initially in the progressive form. A clinically isolated syndrome is the first neurologic episode, which is caused by inflammation/demyelination at one or more sites in the CNS. Progressive-relapsing multiple sclerosis (PRMS) is a rare form of MS (˜5%) characterized by a steadily worsening disease state from onset, with acute relapses but no remissions.

Myelin: A lipid substance forming a sheath (known as the myelin sheath) around the axons of certain nerve fibers. Myelin is an electrical insulator that serves to speed the conduction of nerve impulses in nerve fibers. “Myelination” (also “myelinization”) refers to the development or formation of a myelin sheath around a nerve fiber (axon). Similarly, “remyelination” (also, “remyelinization”) refers to the repair or reformation of the myelin sheath, such as following injury, exposure to a toxic agent, or an inflammatory response, or during the course of a demyelinating disease.

Neurodegenerative disease: Refers to any type of disease that is characterized by the progressive deterioration of the nervous system.

Neuropathy: A functional disturbance or pathological change in the peripheral nervous system. Axonal neuropathy refers to a disorder disrupting the normal functioning of the axons.

Paraproteinemic demyelinating polyneuropathy: A type of peripheral neuropathy characterized by auto antibodies directed against myelin associated glycoproteins (MAG). Anti-MAG antibodies inhibit the production of myelin, thereby leading to neuropathy.

Pelizaeus-Merzbacher disease (PMD): A rare central nervous system disorder in which coordination, motor abilities, and intellectual function are delayed to variable extents. The disease is one in a group of genetic disorders collectively known as leukodystrophies.

Peroneal muscular atrophy (PMA): A genetically and clinically heterogeneous group of inherited disorders of the peripheral nervous system characterized by progressive loss of muscle tissue and touch sensation across various parts of the body. This disease is also known as Charcot-Marie-Tooth disease (CMT), Charcot-Marie-Tooth neuropathy and hereditary motor and sensory neuropathy (HMSN).

Pharmaceutical composition: A composition containing fenfluramine, or a pharmaceutically acceptable salt thereof, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup) or a flexible-dosing, oral solution; for topical administration (e.g., as a cream, gel, lotion, or ointment); for intravenous administration (e.g., as a sterile solution free of particulate emboli and in a solvent system suitable for intravenous use); or in any other formulation described herein.

Pharmaceutically acceptable salt: A salt of fenfluramine which is, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of fenfluramine or separately by reacting the amine group with a suitable acid. and the like. For example, U.S. Pat. No. 10,351,509 describes a synthesis of fenfluramine and pharmaceutically acceptable salts thereof.

Pharmaceutically acceptable excipient (pharmaceutically acceptable carrier): Any ingredient other than fenfluramine, or a pharmaceutically acceptable salt thereof (e.g., a vehicle capable of suspending or dissolving the active compound) and having the properties of being nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

Preventing, treating or ameliorating a disease: “Preventing” refers to a prophylactic treatment or treatment that prevents one or more symptoms or conditions of a disease, disorder, or conditions described herein. Preventive treatment that includes administration of fenfluramine, or a pharmaceutically acceptable salt thereof, or a pharmaceutical composition thereof, can be acute, short-term, or chronic. The doses administered may be varied during the course of preventative treatment. “Treating” refers to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating (palliating)” a disease or condition means that the extent and/or undesirable clinical manifestations of the disease, disorder, or condition are lessened and/or time course of the progression is slowed or lengthened, as compared to the extent or time course in the absence of treatment.

Progressive multifocal leukoencephalopathy (PML): A rare and usually fatal viral disease that is characterized by progressive damage or inflammation of the white matter of the brain in multiple locations. PML occurs almost exclusively in people with severe immune deficiency. The cause of PML is a type of polyomavirus called the JC virus. The virus is widespread, with 86% of the general population presenting antibodies, but it usually remains latent, causing disease only when the immune system has been severely weakened. PML is a demyelinating disease, in which the myelin sheath covering the axons of nerve cells is gradually destroyed, impairing the transmission of nerve impulses. The disease may occur in subjects (e.g., humans) with severe immune deficiency, such as transplant patients on immunosuppressive medications or those receiving certain kinds of medications. For example, PML has been associated with administration of rituximab (off-label use in the treatment of multiple sclerosis). It affects the white matter, which is mostly composed of axons from the outermost parts of the brain (cortex). Symptoms include weakness or paralysis, vision loss, impaired speech, and cognitive deterioration.

Subject: An animal (e.g., a mammal, such as a human) A subject to be treated according to the methods described herein may be one who has been diagnosed with a neurodegenerative disease involving demyelination, insufficient myelination, or under-development of a myelin sheath, e.g., a subject diagnosed with multiple sclerosis or cerebral palsy, or one at risk of developing the condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

Therapeutically effective amount: A quantity of fenfluramine, or a pharmaceutically acceptable salt thereof, sufficient to achieve a desired effect in a subject, or in a cell, being treated with fenfluramine The effective amount of fenfluramine depends on several factors, including, but not limited to the subject or cells being treated, and the manner of administration of the therapeutic composition. In some embodiments, a “therapeutically effective amount” of fenfluramine, or a pharmaceutically acceptable salt thereof, is the amount sufficient to promote myelination in a subject. In other embodiments, a “therapeutically effective amount” of fenfluramine, or a pharmaceutically acceptable salt thereof, is the amount sufficient to inhibit demyelination in a subject.

Transverse myelitis: A neurological disorder caused by an inflammatory process of the grey and white matter of the spinal cord, leading to axonal demyelination. Demyelination arises idiopathically following infections or vaccination, or due to multiple sclerosis. Symptoms include weakness and numbness of the limbs as well as motor, sensory, and sphincter deficits. Severe back pain may occur in some patients at the onset of the disease.

Tropical spastic paraparesis (TSP): An infection of the spinal cord by human T-lymphotropic virus resulting in paraparesis, weakness of the legs. TSP is also known as HTLV-associated myelopathy or chronic progressive myelopathy. As the name suggests, this disease is most common in tropical regions, including the Caribbean and Africa.

Van der Knaap disease: A form of hereditary CNS demyelinating disease. This disease is a type of leukodystrophy and is also known as megalencephalic leukoencephalopathy with subcortical cysts (MLC).

X-linked adrenoleukodystrophy (X-ALD, ALD, or X-linked ALD): A rare, inherited metabolic disorder that leads to progressive brain damage, mental deterioration, failure of the adrenal glands, muscle spasms, blindness and eventually death. ALD is one disease in a group of inherited disorders called leukodystrophies. Adrenoleukodystrophy progressively damages myelin. X-linked ALD male patients may be divided into 7 phenotypes: childhood cerebral (progressive neurodegenerative decline leading to a vegetative state), adolescent (similar to childhood cerebral form but with a slower progression), adrenomyeloneuropathy (progressive neuropathy, paraparesis, may progress to cerebral involvement), adult cerebral (dementia, similar progression to childhood cerebral form), olivo-ponto-cerebellar (cerebral and brain stem involvement), Addison disease (adrenal insufficiency), asymptomatic (no clinical presentation, subclinical adrenal insufficiency, or AMN phenotype). X-linked ALD female patients may be divided into 5 phenotypes: asymptomatic (no neurologic or adrenal involvement), mild myelopathy, moderate to severe myelopathy (similar to male AMN phenotype), cerebral (progressive dementia and decline), and adrenal (primary adrenal insufficiency). X-linked ALD patients may progress from one phenotype to another over the course of their life. ALD is also known as Addison-Schilder disease or Siemerling-Creutzfeldt disease.

Zellweger syndrome: A rare congenital disorder, characterized by the reduction or absence of functional peroxisomes in the cells of an individual. This disease is classified as a leukodystrophy and is one of three peroxisome biogenesis disorders that belong to the Zellweger spectrum of peroxisome biogenesis disorders.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. In case of conflict, the present specification, including explanations of terms, will control. In addition, the materials, methods, and examples are illustrative only and not intended to be limiting.

III. Overview of Several Embodiments

Fenfluramine, or a pharmaceutically acceptable salt thereof, provides a treatment for slowing or halting demyelination and promoting myelination in diseases or conditions associated with demyelination, insufficient myelination, or underdevelopment of the myelin sheath.

The present disclosure features a method of treating a subject having or at risk of developing X-linked adrenoleukodystrophy by administering to the subject in need thereof a therapeutically effective amount of fenfluramine or a pharmaceutically acceptable salt thereof. The present disclosure also features a method of inhibiting accumulation of very-long chain fatty acids in a cell in a patient that has or is at risk of developing X-linked adrenoleukodystrophy, by contacting the neuron with fenfluramine, or a pharmaceutically acceptable salt thereof.

In some embodiments, the phenotype of X-linked adrenoleukodystrophy is childhood cerebral, adolescent, adrenomyeloneuropathy, adult cerebral, olivo-ponto-cerebellar, Addison disease, or asymptomatic. In other embodiments, the phenotype of X-linked adrenoleukodystrophy is asymptomatic, mild myelopathy, moderate to severe myelopathy (e.g., adrenomyeloneuropathy), cerebral, and adrenal. In certain embodiments, the phenotype of X-linked adrenoleukodystrophy is cerebral. In other embodiments, the phenotype of X-linked adrenoleukodystrophy is myelopathy (e.g., moderate to severe myelopathy). In certain other embodiments, the phenotype of X-linked adrenoleukodystrophy is asymptomatic. In yet other embodiments, the phenotype of X-linked adrenoleukodystrophy is Addison disease. In certain embodiments, the phenotype of X-linked adrenoleukodystrophy is olivo-ponto-cerebellar.

The present disclosure features a method of treating a subject having or at risk of developing a disease or condition associated with demyelination, insufficient myelination, or underdevelopment of myelin sheath. The method involves administration of a therapeutically effective amount of fenfluramine, or a pharmaceutically acceptable salt thereof.

The present disclosure features a method of inhibiting demyelination of a neuron in a patient that has or is at risk of developing a disease or condition associated with demyelination, insufficient myelination, or underdevelopment of myelin sheath, by contacting the neuron with fenfluramine, or a pharmaceutically acceptable salt thereof.

The present disclosure also features a method of promoting myelination of a neuron in a patient that has or is at risk of developing a disease or condition associated with demyelination, insufficient myelination, or underdevelopment of myelin sheath, by contacting the neuron with fenfluramine, or a pharmaceutically acceptable salt thereof.

The disease or condition to be treated can be any disease or condition associated with demyelination, insufficient myelination or underdevelopment of myelin sheath. In some embodiments, the disease or condition is multiple sclerosis, a leukodystrophy, a leukoencephalopathy, an idiopathic inflammatory demyelinating disease, or Alzheimer's disease. In some examples in which the disease or condition is multiple sclerosis, the multiple sclerosis is relapsing-remitting multiple sclerosis, primary-progressive multiple sclerosis, secondary-progressive multiple sclerosis, or progressive-relapsing multiple sclerosis.

In some embodiments, the disease or condition is central pontine myelinolysis, acute disseminated encephalomyelitis, Balo concentric sclerosis, Marburg multiple sclerosis, tumefactive multiple sclerosis, diffuse myelinoclastic sclerosis, acute hemorrhagic leukoencephalitis, neuromyelitis optica, a chronic inflammatory demyelinating polyneuropathy, Leber hereditary optic neuropathy, multifocal motor neuropathy, paraproteinemic demyelinating polyneuropathy, tropical spastic paraparesis, a Guillain-Barre syndrome, infantile Refsum disease, adult Refsum disease 1, adult Refsum disease 2, Zellweger syndrome, X-linked adrenoleukodystrophy (X-ALD), metachromatic leukodystrophy, Krabbe disease, Pelizaeus-Merzbacher disease, Canavan disease, Alexander disease, Binswanger's disease, peroneal muscular atrophy, cerebrotendinous xanthomatosis, leukoencephalopathy with vanishing white matter, toxic leukoencephalopathy, van der Knaap disease, progressive multifocal leukoencephalopathy. Marchiafava-Bignami disease or transverse myelitis.

In some examples, the Guillain-Barre syndrome is acute inflammatory demyelinating polyneuropathy.

In some examples, the chronic inflammatory demyelinating polyneuropathy is multifocal acquired demyelinating sensory and motor neuropathy. In some examples, the chronic inflammatory demyelinating polyneuropathy is induced by HIV infection.

In some embodiments, the disease or condition is a chronic axonal neuropathy.

In some embodiments, the disease or condition results from intraventricular hemorrhage, neonatal hypoxia, or acute hypoxemic respiratory failure.

In some embodiments, the disease or condition is cerebral palsy.

In some embodiments of the disclosed method, administration of the fenfluramine or pharmaceutically acceptable salt thereof prevents or mitigates at least one symptom of the disease or condition. In some examples, the symptom is a lack of sphincter control, erectile dysfunction, paraparesis, ataxia, adrenocortical insufficiency, progressive neuropathy, paresthesia, dysarthria, dysphagia, clonus, or any combination thereof.

In some embodiments, administration of the fenfluramine or pharmaceutically acceptable salt thereof prevents or mitigates damage to central nervous system myelin, peripheral nervous system myelin, adrenal cortex, testicular Leydig cells, or any combination thereof.

In certain embodiments, fenfluramine, or a pharmaceutically acceptable salt thereof, is administered orally, parenterally, or topically. In particular embodiments, fenfluramine, or a pharmaceutically acceptable salt thereof, is administered orally. In certain embodiments, fenfluramine, or a pharmaceutically acceptable salt thereof, is administered enterally. In some embodiments, fenfluramine, or a pharmaceutically acceptable salt thereof, is administered buccally, sublingually, sublabially, or by inhalation. In other embodiments, fenfluramine, or a pharmaceutically acceptable salt thereof, is administered sublingually. In yet other embodiments, fenfluramine, or a pharmaceutically acceptable salt thereof, is administered parenterally. In particular embodiments, fenfluramine, or a pharmaceutically acceptable salt thereof, is administered intra-arterially, intravenously, intraventricularly, intramuscularly, subcutaneously, intraspinally, intraorbitally, intracranially or intrathecally.

In some embodiments, the fenfluramine or pharmaceutically acceptable salt thereof is administered at a dose of about 0.5 mg/kg/day to about 5 mg/kg/day. In some examples, fenfluramine or a pharmaceutically acceptable salt thereof is administered at a dose oral dose of fenfluramine or a pharmaceutically acceptable salt thereof to the patient in an amount in a range of 0.2 mg/kg/day to 0.8 mg/kg/day up to a maximum of 30 mg/day In some embodiments, the fenfluramine or pharmaceutically acceptable salt thereof is administered daily.

In particular embodiments, the compound is administered to the subject once daily, twice daily, three times daily, once every two days, once weekly, twice weekly, three times weekly, once biweekly, once monthly, or once bimonthly. In certain embodiments, the compound is administered to the subject once daily. In other embodiments, the effective amount in an oral dose of fenfluramine or a pharmaceutically acceptable salt thereof to the patient is an amount in a range of 0.2 mg/kg/day to 0.8 mg/kg/day up to a maximum of 30 mg/day.

In some embodiments, the methods of the present disclosure involve administering a unit dosage form containing from an amount in a range of 0.2 mg/kg/day to 0.8 mg/kg/day up to a maximum of 30 mg/day fenfluramine, or a pharmaceutically acceptable salt thereof, once, twice or three times per day. In some embodiments, the methods of the present disclosure involve administering a unit dosage form containing of fenfluramine, or a pharmaceutically acceptable salt thereof, once, twice or three times per day. In other embodiments, the methods of the present disclosure involve administering a unit dosage form containing from of fenfluramine, or a pharmaceutically acceptable salt thereof, once, twice or three times per day. In particular embodiments, the methods of the present disclosure involve administering a unit dosage form containing from of fenfluramine, or a pharmaceutically acceptable salt thereof, once, twice or three times per day. In yet other embodiments, the methods of the present disclosure involve administering a unit dosage form containing from of fenfluramine, or a pharmaceutically acceptable salt thereof, once, twice or three times per day. In still other embodiments, the methods of the present disclosure involve administering a unit dosage form containing of fenfluramine, or a pharmaceutically acceptable salt thereof, once, twice or three times per day.

The present disclosure also features a method of treating a patient having or at risk of developing multiple sclerosis by administering to the patient a therapeutically effective amount of fenfluramine, or a pharmaceutically acceptable salt thereof. In one example, provided is a method of treating a patient having or at risk of developing multiple sclerosis, comprising administering to the patient 1 mg/kg of the weight of said patient/day of fenfluramine, or a pharmaceutically acceptable salt thereof.

Administration of fenfluramine and pharmaceutically acceptable salts thereof is further discussed in the section below.

IV. Administration of Fenfluramine or Pharmaceutical Compositions Thereof

Fenfluramine and pharmaceutically acceptable salts thereof can be administered according to any suitable route of administration for the treatment of a disease or condition associated with demyelination, insufficient myelination, or underdevelopment of myelin sheath. For example, standard routes of administration include oral, parenteral, or topical routes of administration. In particular, the route of administration of fenfluramine or a pharmaceutically acceptable salt thereof may be oral (e.g., enteral, buccal, sublingual, sublabial, or by inhalation). Parenteral route of administration of fenfluramine, or a pharmaceutical composition thereof, may be, e.g., intra-arterial, intravenous, intraventricular, intramuscular, subcutaneous, intraspinal, intraorbital, or intracranial. Topical route of administration may be, e.g., cutaneous, intranasal, or ophthalmic.

Pharmaceutical compositions comprising fenfluramine have been described in the art (see, e.g., U.S. Pat. No. 5,883,294, which is herein incorporated by reference).

Fenfluramine and pharmaceutically acceptable salts thereof that are to be administered orally can be formulated as liquids, for example syrups, suspensions or emulsions, or as tablets, capsules or lozenges.

A liquid composition will generally include a suspension or solution of fenfluramine or pharmaceutically acceptable salt in a suitable liquid carrier, for example ethanol, glycerin, sorbitol, non-aqueous solvent such as polyethylene glycol, oils or water, with a suspending agent, preservative, surfactant, wetting agent, flavoring or coloring agent. Alternatively, a liquid formulation can be prepared from a reconstitutable powder, i. e. a liquid which is reconstituted by adding water to a powder.

In some cases, a powder containing active compound, suspending agent, sucrose and a sweetener can be reconstituted with water to form a suspension; and a syrup can be prepared from a powder containing active ingredient, sucrose and a sweetener.

A composition in the form of a tablet can be prepared using any suitable pharmaceutical carrier(s) routinely used for preparing solid compositions. Examples of such carriers include magnesium stearate, starch, lactose, sucrose, microcrystalline cellulose and binders, for example polyvinylpyrrolidone. The tablet can also be provided with a color film coating, or color included as part of the carrier(s). In addition, active compound can be formulated in a controlled release dosage form as a tablet comprising a hydrophilic or hydrophobic matrix.

A composition in the form of a capsule can be prepared using routine encapsulation procedures, for example by incorporation of active compound and excipients into a hard gelatin capsule. Alternatively, a semi-solid matrix of active compound and high molecular weight polyethylene glycol can be prepared and filled into a hard gelatin capsule; or a solution of active compound in polyethylene glycol or a suspension in edible oil, for example liquid paraffin or fractionated coconut oil can be prepared and filled into a soft gelatin capsule. Fenfluramine and pharmaceutically acceptable salts thereof to be administered parenterally can be formulated, for example, for intramuscular or intravenous administration.

In some instances, a composition for intramuscular administration contains a suspension or solution of active ingredient in an oil, for example arachis oil or sesame oil. A composition for intravenous administration can include a sterile isotonic aqueous solution containing, for example active ingredient, dextrose, sodium chloride, a co-solvent, for example polyethylene glycol and, optionally, a chelating agent, for example ethylenediamine tetracetic acid and an anti-oxidant, for example, sodium metabisulphite. Alternatively, the solution can be freeze dried and then reconstituted with a suitable solvent just prior to administration.

Fenfluramine and pharmaceutically acceptable salts thereof for rectal administration can be formulated as suppositories. A typical suppository formulation will generally include active ingredient with a binding and/or lubricating agent such as a gelatin or cocoa butter or other low melting vegetable or synthetic wax or fat.

Fenfluramine and pharmaceutically acceptable salts thereof to be administered topically can be formulated as transdermal compositions. Such compositions include, for example, a backing, active compound reservoir, a control membrane, liner and contact adhesive.

Non-limiting examples of formulations for buccal, sublingual, and/or sublabial administration may be found in U.S. Pre-grant Publication No. 2012/0058962, U.S. Pre-grant Publication No. 2013/0225626, U.S. Pre-grant Publication No. 2009/0117054, and U.S. Pat. No. 8,252,329; the disclosure of each of which is incorporated herein by reference.

For buccal, sublingual, or sublabial administration, the compositions may take the form of tablets, lozenges, etc. formulated in a conventional manner, as described for oral dosage forms. In some embodiments, the formulation for buccal, sublingual, or sublabial administration includes one or more of taste masking agents, enhancers, complexing agents, and other described above pharmaceutically acceptable excipients and carriers.

Taste masking agents include, for example, taste receptor blockers, compounds which mask the chalkiness, grittiness, dryness, and/or astringent taste properties of an active compound, compounds which reduce throat catch as well as compounds which add a flavor. A taste receptor blocker used in the formulation of the present disclosure may include Kyron T-134, a glycoprotein extract called miraculin from the fruit of the plant synsepalum dulcifcum, ethyl cellulose, hydroxypropyl methylcellulose, arginine, sodium carbonate, sodium bicarbonate, gustducin blockers and mixtures thereof. Compounds which mask the chalkiness, grittiness, dryness and/or astringent taste properties of an active compound include those of a natural or synthetic fatty type or other flavorant such as cocoa, chocolate (e.g., mint chocolate), cocoa butter, milk fractions, vanillin butter fat, egg or egg white, peppermint oil, wintergreen oil, spearmint oil, and similar oils. Compounds which reduce throat catch include combinations of high and low solubility acids. For example, high solubility acids suitable for use here include amino acids (e.g., alanine, arginine etc.), glutaric, ascorbic, malic, oxalic, tartaric, malonic, acetic, citric acids and mixtures thereof. Low solubility acids suitable for use include oleic, stearic and aspartic acids plus certain amino acids such as glutamic acid, glutamine, histidine, isoleucine, leucine, methionine, phenylalanine, serine, tryptophan, tyrosine, valine and fumaric acid. Actual amounts used will vary depending on the amount of throat catch or burn exhibited by the active used but will generally be in the range of 1 to 40%. Flavoring agents include sweeteners and flavors. Examples of suitable sweeteners and flavors include mannitol, sorbitol, maltitol, lactitol, isomaltitol, erythritol, xylitol, sucrose, ammonium glycyrrhizinate, mango aroma, black cherry aroma, sodium citrate, colloidal silicon dioxide, sucralose; zinc gluconate; ethyl maltitol; glycine; acesulfame-K; aspartame; saccharin; acesulfam K, neohesperidin DC, thaumatin, stevioside, fructose; xylitol; honey; honey extracts; corn syrup, golden syrup, misri, spray dried licorice root; glycerrhizine; dextrose; sodium gluconate; stevia powder; glucono delta-lactone; ethyl vanillin; vanillin; normal and high-potency sweeteners or syrups or salts thereof and mixtures thereof. Other examples of appropriate flavoring agents include coffee extract, mint; lamiacea extracts; citrus extracts; almond oil; babassu oil; borage oil; blackcurrant seed oil; canola oil; castor oil; coconut oil; corn oil; cottonseed oil; evening primrose oil; grape seed oil; groundnut oil; mustard seed oil; olive oil; palm oil; palm kernel oil; peanut oil; grapeseed oil; sunflower oil; sesame oil; shark liver oil; soybean oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides; linoleic glycerides; caprylic/capric glycerides; modified triglycerides; fractionated triglycerides; safrole, citric acid, d-limonene, malic acid, and phosphoric acid or salts and/or mixtures thereof.

Enhancers are the agents that increase membrane permeability and/or increase the solubility of a particular active compound. Both issues can be pivotal to the properties of the formulation. An enhancer may be a chelator, a surfactant, a membrane-disrupting compound, a fatty or other acid; a non-surfactant, such as an unsaturated cyclic urea. A chelator may be, e.g., EDTA, citric acid, sodium salicylate, or a methoxysalicylate. A surfactant may be, e.g., sodium lauryl sulphate, polyoxyethylene, POE-9-laurylether, POE-20-cetylether, benzalkonium chloride, 23-lauryl ether, cetylpyridinium chloride, cetyltrimethyl ammonium bromide, or an amphoteric or a cationic surfactant. A membrane-disrupting compound may be, e.g., a powdered alcohol (such as, menthol) or a compound used as lipophilic enhancer. Fatty and other acids include, e.g., oleic acid, capric acid, lauric acid, lauric acid/propylene glycol, methyloleate, lyso-phosphatidylcholine, and phosphatidylcholine. Other enhancers that may be used in buccal, sublingual, and sublabial formulations of the present disclosure include, e.g., lysalbinic acid, glycosaminoglycans, aprotinin, azone, cyclodextrin, dextran sulfate, curcumin, menthol, polysorbate 80, sulfoxides, various alkyl glycosides, chitosan-4-thiobutylamide, chitosan-4-thiobutylamide/GSH, chitosan-cysteine, chitosan-(85% degree N-deacetylation), poly(acrylic acid)-homocysteine, polycarbophil-cysteine, polycarbophil-cysteine/GSH, chitosan thioethylamide/GSH, chitosan-4-thioglycholic acid, hyaluronic acid, propanolol hydrochloride, bile salts, sodium glycocholate, sodium deoxycholate, sodium taurocholate, sodium glycodeoxycholate, and sodium taurodeoxycholate.

Buffering materials can be both used to increase solubility and enhance adsorption of active compounds. Examples of suitable buffering materials or antacids suitable for use herein comprise any relatively water soluble antacid acceptable to the Food & Drug Administration, such as aluminum carbonate, aluminum hydroxide (or as aluminum hydroxide-hexitol stabilized polymer, aluminum hydroxide-magnesium hydroxide co-dried gel, aluminum hydroxide-magnesium trisilicate codried gel, aluminum hydroxide-sucrose powder hydrated), aluminum phosphate, aluminum hydroxyl carbonate, dihydroxyaluminum sodium carbonate, aluminum magnesium glycinate, dihydroxyaluminum aminoacetate, dihydroxyaluminum aminoacetic acid, bismuth aluminate, bismuth carbonate, bismuth subcarbonate, bismuth subgallate, bismuth subnitrate, calcium carbonate, calcium phosphate, hydrated magnesium aluminate activated sulfate, magnesium aluminate, magnesium aluminosilicates, magnesium carbonate, magnesium glycinate, magnesium hydroxide, magnesium oxide, and magnesium trisilicate, and/or mixtures thereof. Preferred buffering materials or antacids include aluminum hydroxide, calcium carbonate, magnesium carbonate and mixtures thereof, as well as magnesium hydroxide. Many of these compounds have the advantage of also being taste masking agents particularly useful for addressing throat catch.

The selection of the other excipients, such as permeation enhancers, disintegrants, masking agents, binders, flavors, sweeteners and taste-masking agents, is specifically matched to the active depending on the predetermined pharmacokinetic profile and/or organoleptic outcome

Liquid drug formulations suitable for use with nebulizers and liquid spray devices and electrohydrodynamic (EHD) aerosol devices will typically include fenfluramine or a pharmaceutically acceptable salt thereof with a pharmaceutically acceptable carrier. Preferably, the pharmaceutically acceptable carrier is a liquid, e.g., alcohol, water, polyethylene glycol, or a perfluorocarbon. Optionally, another material may be added to alter the aerosol properties of the solution or suspension. Desirably, this material is liquid, e.g., an alcohol, glycol, polyglycol, or a fatty acid. Other methods of formulating liquid drug solutions or suspension suitable for use in aerosol devices are known to those of skill in the art (see, e.g., U.S. Pat. Nos. 5,112,598 and 5,556,611, each of which is herein incorporated by reference).

The dose and dosing schedule for administration of fenfluramine (or a pharmaceutically acceptable salt thereof) can vary and is determined in part by the severity of the disease, and the age, weight and general health of the patient. In some embodiments, the composition is administered daily. In other embodiments the composition is administered more than once a day, such as twice a day, three time a day or four times a day. In yet other embodiments, the composition is administered less than once a day, such as every other day, every three days or once a week.

In some embodiments of the methods, the dose of fenfluramine (or a pharmaceutically acceptable salt thereof) may be about 0.1 mg/kg/day to about 3 mg/kg/day. (e.g., twice daily, once daily, twice weekly, or once weekly the dose of fenfluramine (or a pharmaceutically acceptable salt thereof) or may be about 0.1 mg/kg/day to about 2 mg/kg/day. (e.g., twice daily, once daily, twice weekly, or once weekly). In some embodiments, the dose of fenfluramine (or a pharmaceutically acceptable salt thereof) is capped at no more the 60 mg/day. In particular examples, the dose of fenfluramine (or a pharmaceutically acceptable salt thereof) is about (e.g., twice daily, once daily, twice weekly, or once weekly).

V. Diagnosing

As described above, provided is a method of treatment comprising administering to a subject diagnosed with a demyelination disorder a therapeutically effective amount of fenfluramine or a pharmaceutically acceptable salt thereof. Also provided are methods that involve administering to a subject diagnosed with a condition associated with demyelination.

In some cases, the methods include the step of diagnosing the subject with the demyelination disorder or the condition associated with demyelination. In some cases, the subject was already diagnosed before the method.

In some embodiments, the subject is diagnosed based on data selected from the group consisting of a magnetic resonance image (MRI), electromyography (EMG), a nerve conductivity study (NCV), an evoked potential study, a lumbar puncture (LP), history of pain, history of nausea, history of vomiting, history of fever, muscle strength, nerve sensation, motor coordination, ability to walk, an eye exam, or a combination thereof. For instance, such tests and the resulting data can be used to diagnose the patient with a demyelination disorder.

The magnetic resonance image (MRI) can show features of the brain or spine, such as a two-dimensional or three-dimensional image of such organs. Areas of demyelination can be observed on the MRI, and can also be used to determine the type of demyelination. For example, Miki describes methods of diagnosing a demyelination disease using MRI (doi:10.111/cen3.12501) and is incorporated herein by reference. Miki states that MRI can be used to differentiate between and diagnose different types of demyelinating diseases, including multiple sclerosis (MS), neuromyelitis optica spectrum disorder (NMOSD), acute disseminated encephalomyelitis (ADEM) and myelin oligodendrocyte glycoprotein (MOG) encephalomyelitis.

Electromyography (EMG) can involve inserting thin needle electrodes through the skin and into muscles to measure muscle activity during movement and during a resting state. Such electrical readings can be used to diagnose a disorder. Nerve conduction studies (NCV) measure the speed at which nerves conduct electrical signals, such as by electrically stimulating a nerve with an electrode inserted into the skin and to a nerve. Evoked potential studies measure the brain response to certain stimuli, e.g. the brain's response to visual input. Lumbar puncture (LP), which is also referred to as a spinal tap, involves removing cerebrospinal fluid (CSF), such as from the lower back near the spinal cord. Such fluid can be analyzed to determine possible infections or inflammatory conditions.

A patient's medical history can be considered in making a diagnosis. For instance, the patient's history of pain, nausea, vomiting, and fevers can be used to make a diagnosis. The history of such conditions in a patient's biological family can also be considered.

Diagnosing can involve assessing a patient's muscle strength, such as the maximum force they can exert. In some cases, nerve sensation, i.e. the ability of the nerves to detect stimuli. Exemplary stimuli include pressure, cold temperature, hot temperature, and pain. Such nerve sensation studies can, in some cases, be performed on the skin of the subject. In some embodiments the diagnosing includes assessing the subject's motor coordination, which is their ability to move multiple body parts in a concerted manner Such concerted motions can be, in some cases, walking, running, standing up, sitting down, or grabbing an object with a hand. In some embodiments, diagnosing involves performing an eye exam of the subject. In some cases, inflammation of the optic nerve (i.e. optic neuritis) can be observed during an eye exam, which can be caused by demyelination or other factors.

The following examples are provided to illustrate certain particular features and/or embodiments. These examples should not be construed to limit the disclosure to the particular features or embodiments described.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); and the like.

### Example 1

### Fenfluramine Binds to RNF43

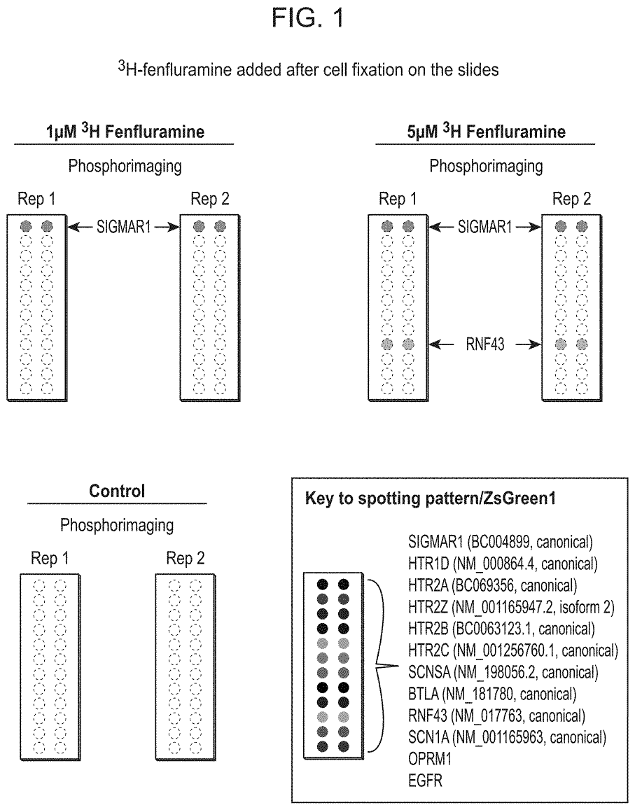

Briefly, arrays of expression vectors, encoding full-length human plasma membrane and tethered secreted proteins, were spotted onto coated slides, then HEK293 cells were grown over the top of the spots and became reverse-transfected resulting in cell surface expression of each respective protein at distinct slide locations. Tritium labeled fenfluramine was applied and specific binding analyzed and confirmed using a phosphorimaging technique. RNF43 and sigma-1 interactions were identified in the primary screen. Confirmation was achieved by repeating the study with vectors encoding RNF43 and sigma-1 receptor proteins spotted onto slides, upon which HEK293 cells were reverse-transfected. Identical slides were treated before or after cell fixation with 1 micromolar or 5 micromolar 3H-fenfluramine, 20 nanomolar 3H-naloxone, or no compound (n=2 slides per treatment). Results are shown in FIG. 1. Low intensity interactions with test compounds in this screen do not necessarily indicate low affinity interactions, particularly on receptors not preferentially located on the cell surface.

Epilepsy is increasingly recognized as a disease of white matter pathology and damage, see Hatton, S. N. et al [Epilepsy Currents 2021 21:27-29]. To test whether fenfluramine reduced pathology and damage to myelin, a mouse model of Dravet syndrome was used. Mouse models of Dravet have been shown to have myelin pathology and damage, for example, see Richards, K. et al [Brain Research 2021 January; 1751:147157]. Human Dravet patients have also been shown to have myelin pathology and damage, for example Perez, A. et al. [Epilepsy Res. 2014 108:1326-1334]. Heterozygous Dravet animals used in the studies reported in Examples 2 and 3 are Scn1atm1Kea 50% C57BL/6J, 50% 129S6/SvEvTac background strain mice generated as described in Miller, A. R. et al. [Genes Brain Behav. 2014 February; 13(2): 163-172]. Briefly, a heterozygous Scn1a+/− null allele was generated by targeted deletion of the first coding exon in TL1 ES cells (12956/SvEvTac). These mice were established and maintained as a co-isogenic strain on the 12956/SvEvTac (129) background. Crossing 129.Scn1a+/− mice to strain B6 resulted in (B6x129)FEScn1a+/− (FEScn1a+/−) offspring that exhibit spontaneous seizures and premature lethality, with 50% of mice dying by 1 month of age. P ranges, stated as, e.g. P35-P37, indicate the days postnatal of the study animals Normal axonodendritic, axonosomatic and axonoaxonic synaptic pruning occurs in all mice and peaks at postnatal P21. Postnatal ages for the study animals were chosen to avoid periods of high natural demyelination and microglial activity.

### Example 2

Use of Fenfluramine for the Treatment of Demyelination

Fenfluramine Decreased Demyelination in a Murine model of Dravet syndrome

A study of the effects of fenfluramine, a compound known to have efficacy in treatment of Dravet syndrome in humans, and diazepam, a compound also known to have activity in suppression of seizures were compared to demonstrate that fenfluramine's myelination activity is independent of its seizure reducing properties. In Heterozygous Animals, drug or saline sham treatment began at age P7 (i.e., 7 days after birth) with subcutaneous administration of drug or saline injected via insulin syringes.

At day 7 all pups in each litter were administered an injection volume of 10 μl/g body weight once per day containing: 1) fenfluramine at 15 mg/kg; 2) diazepam at 10 mg/kg; or 3) vehicle (0.9% saline solution), each injection volume based on body weight.

The administration of each intervention continued every day until age P35-P37, at which time animals were sacrificed to obtain blood and brain tissues which amounted to an aggregate of 28 to 30 days of once daily dosing.

Mice at the end of the dosing period were isoflurane anaesthetized and blood was drawn through cardiac ventricular puncture without perforating the heart or vessels for subsequent perfusion fixation. Blood was processed for plasma and stored at −80C. After creating an incision in the hepatic circulation to allow perfusates an exit point, mice were flushed of remaining blood with 6 mL ice cold phosphate buffered saline through the left ventricle of the heart. Mice were then perfusion fixed with an additional 6 mL of ice cold 4% paraformaldehyde before carefully harvesting right brains into 10% neutral buffered formalin for 24 hours before transferring into phosphate buffered saline with 0.02% sodium azide as an antimicrobial preservative. Right brains were then embedded to paraffin blocks and processed as formalin fixed, paraffin embedded (FFPE) 5 μm thick sagittal sections on glass slides. These FFPE tissues were immunostained, processed, and imaged for degraded myelin and inflammatory microglia.

DMBP antibody (“degraded” Myelin Basic Protein, Anti-Myelin Basic Protein, Millipore Sigma antibody, formerly Sigma AB5864) was applied at 1:1000 dilution with no antigen retrieval. This antibody preferentially detects debris of myelin degradation in human samples without cross reacting to normal myelin. Vector ImPRESS-AP alkaline phosphatase conjugated anti-rabbit IgG (Vector Laboratories) was applied in the secondary detection step with Vector Blue AP substrate as the chromogen. Nuclear Fast Red was applied as a non-specific tissue counterstain before dehydration, clearing, and coverslip mounting steps. Vector Blue signals within finished slides were visualized with the DAPI (UV) excitation/emission channel on an Echo Revolve fluorescence microscope under the 20× objective. Non-specific Nuclear Fast Red staining was visualized under the Texas Red excitation/emission channel to provide anatomical contexts to the overlaid DMBP signals. Perfusion fixed sagittal 5 micrometer (“um”) FFPE brain sections within approximately 300um from the midline of the right brains of the P35-P37 wildtype and Heterozygous Animals from the Fenfluramine/Diazepam/Saline comparator study were examined for myelin damage as indicated by DMBP staining of spheroid and punctate myelin debris.

A 0-5 ascending pathologist scale was applied in scoring the hippocampus and cortex. 0: no spheroid myelin debris (SMD). 1: low but present SMD. 2: noticeably increased SMD. 3: intermediate SMD. 4: high SMD. 5: very high SMD. The scores of the hippocampus and cortex were summed for each animal for a maximum possible score of 10.

In rat CNS tissue, the DMBP antibody detects unambiguous spheroidal and punctate debris of myelin degradation with some intermediate cross reactivity to normal myelin. In mouse CNS tissue, the DMBP antibody detects unambiguous spheroidal and punctate debris of myelin degradation with increased reactivity to normal myelin compared to its performance upon human CNS tissue. Spheroidal and isolated punctate debris represent debris generated by pathological processes such as myelin sheath generation defects, axonopathy, inflammatory demyelination, as well as physiological pruning or pathological destruction of axonodendritic, axonosomatic and axonoaxonic presynaptic myelinated axons.

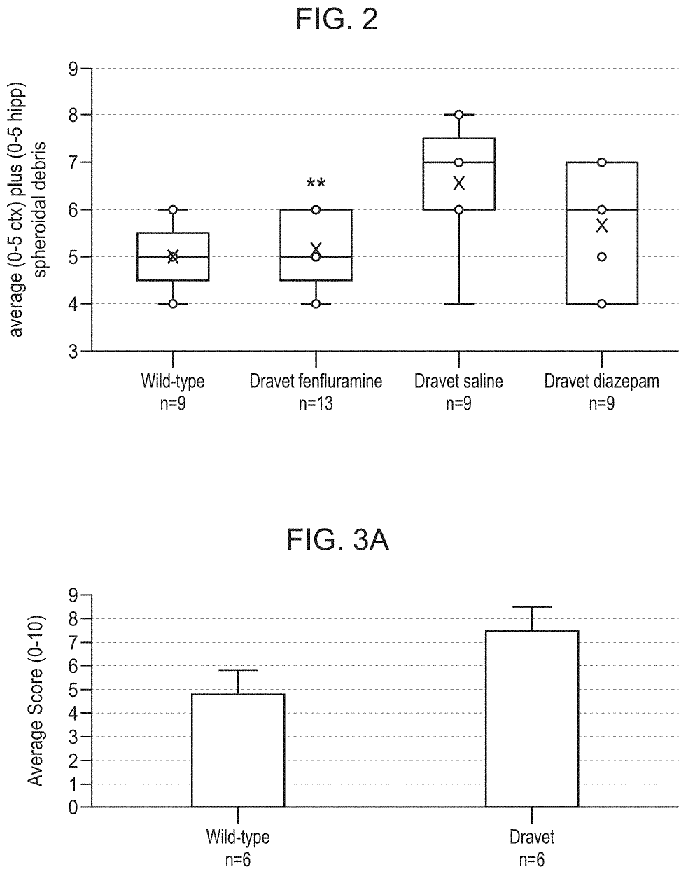

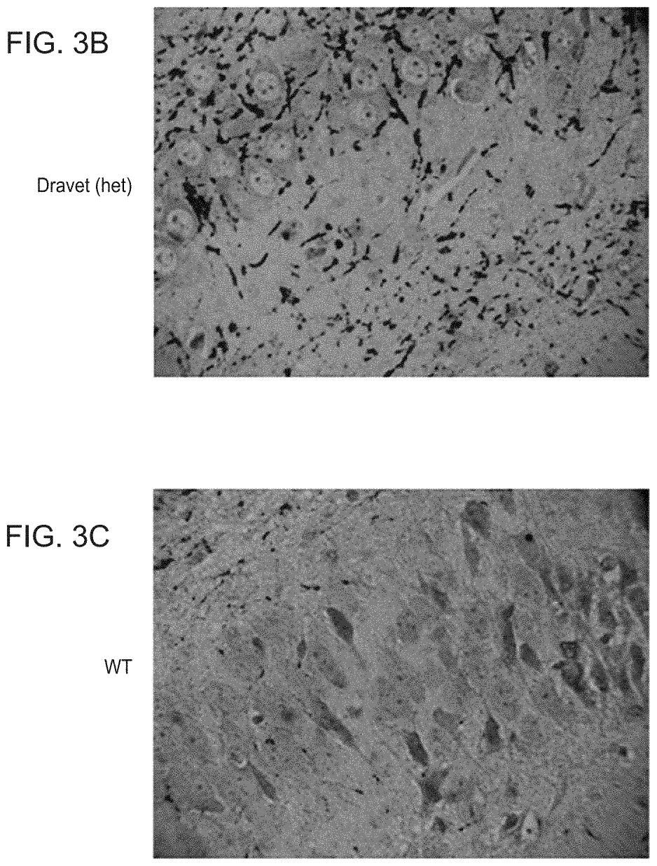

Spheroidal and punctate debris detected by the DMBP antibody are admitted into the data as pathological signals. Visibly intact linear myelin on normal axons cross-detected by DMBP antibody in the rodent tissue was not considered pathological for the ranking scale. Table 1 shows the average scores from the cortex and hippocampus on a scale of 0-5 as described above (ctx=cortex; hipp=hippocampus) and as the sum of the average scores from the two brain tissues. Mice with the Scn1a+/− Dravet mutation are indicated as “Het” and animals without the Dravet mutation (Scn1a+/+) are indicated by “WT”. Fenfluramine-treated animals are abbreviated as “FFA”. P-values were calculated using a two-tailed t-test with unequal variances. These data are shown graphically in FIG. 2. These data show that administration of fenfluramine reduces levels of myelin pathology and damage within the context of a mouse model of a human disease that is known to have myelin damage.

TABLE 1
DMBP scoring and standard deviation

| Intervention | FFA | FFA | Vehicle | Vehicle | Diazepam | Diazepam | FFA | Vehicle | Diazepam |
|---|---|---|---|---|---|---|---|---|---|
| Het mice | Hipp | Ctx | Hipp | Ctx | Hipp | Ctx | Hipp + | Hipp + | Hipp + |
| | | | | | | | Ctx | Ctx | Ctx |
| DMBP | 2.62 | 2.54 | 3.67 | 2.89 | 3.22 | 2.44 | 5.15 | 6.56 | 5.67 |
| Avg Score | | | | | | | | | |
| stdev | 0.51 | 0.66 | 0.50 | 0.93 | 0.83 | 0.73 | 0.80 | 1.24 | 1.41 |
| WT mice | | | Hipp | Ctx | | | | Hipp + | |
| | | | | | | | | Ctx | |
| DMBP | | | 2.89 | 2.11 | | | | 5.0 | |
| Avg Score | | | | | | | | | |
| stdev | | | 0.78 | 0.60 | | | | 0.71 | |

DMBP P-values: Combined Hipp and Ctx p = 0.01 for Het FFA vs Het vehicle
DMBP P-values: Hippocampus only p = 0.00015 Het FFA vs Het vehicle

### Example 3

Fenfluramine Decreases Activated Microglia in a Murine model of Dravet syndrome

Similar to the tissue imaging and counting of myelin debris in Example 2, another study was conducted to show the effect of fenfluramine on microglial activation CD11b antibody (Abcam ab133357) was applied at 1:12000 dilution with pH9 heat-induced antigen retrieval. Vector ImPRESS-AP alkaline phosphatase conjugated anti-rabbit IgG (Vector Laboratories) was applied in the secondary detection step with Vector Blue AP substrate as the chromogen. Nuclear Fast Red was applied as a non-specific tissue counterstain before dehydration, clearing, and coverslip mounting steps. Vector Blue signals within finished slides were visualized with the DAPI (UV) excitation/emission channel on an Echo Revolve fluorescence microscope under the 20× objective. Non-specific Nuclear Fast Red staining was visualized under the Texas Red excitation/emission channel to provide anatomical contexts to the overlaid CD11b signals. Perfusion fixed sagittal 5 um FFPE brain sections within approximately 300 um from the midline of the right brains of the P35-P37 wildtype and SCN1A heterozygous 50% B6/50% 129S6 mice from the Fintepla/Diazepam/Saline comparator study were examined for CD11b+ microglia.

A 0-5 ascending pathologist scale was applied in scoring CD11b+ microglia in the perforant pathway of the hippocampus following the hippocampal fissure and murine white matter in the front knob, “genu,” of the corpus callosum (CC). The CC is an elongated midline structure composed of 200-800 million horizontal interconnecting cortical areas. The mature CC contains both myelinated and unmyelinated fibers as well as glial cells (astrocytes and oligodendrocytes), and neurons. The human CC has been divided into five anatomical regions, which include from front to back, the rostrum, the genu, the body or trunk-often subdivided into anterior, middle and posterior body-the isthmus, and the splenium. Most neurons in the callosal fibers release excitatory amino acids (glutamate or aspartate), however a small proportion of GABAergic neurons have been identified in mammals

Scores were assigned as 0: no activated microglia—1: low numbers of activated microglia—2: noticeably increased activated microglia—3: intermediate activated microglia—4: high activated microglia—5: very high activated microglia. The scores of the genu and perforant pathway were summed for each animal for a maximum possible score of 10. This antibody preferentially detects activated microglia over surveilling, quiescent microglia across human, rat, and mouse species. Normal, healthy mice have more basal microglial activation in the CNS compared to the normal human CNS. Activation is seen as an unambiguous increase in microglial body size, ramification thickness, length when the activation leads to rod shaped microglia, clear phagosomes extending from microglia, or a change to a macrophage phenotype termed “amoeboid.”

Table 2 CD11b+ scoring and standard deviations.

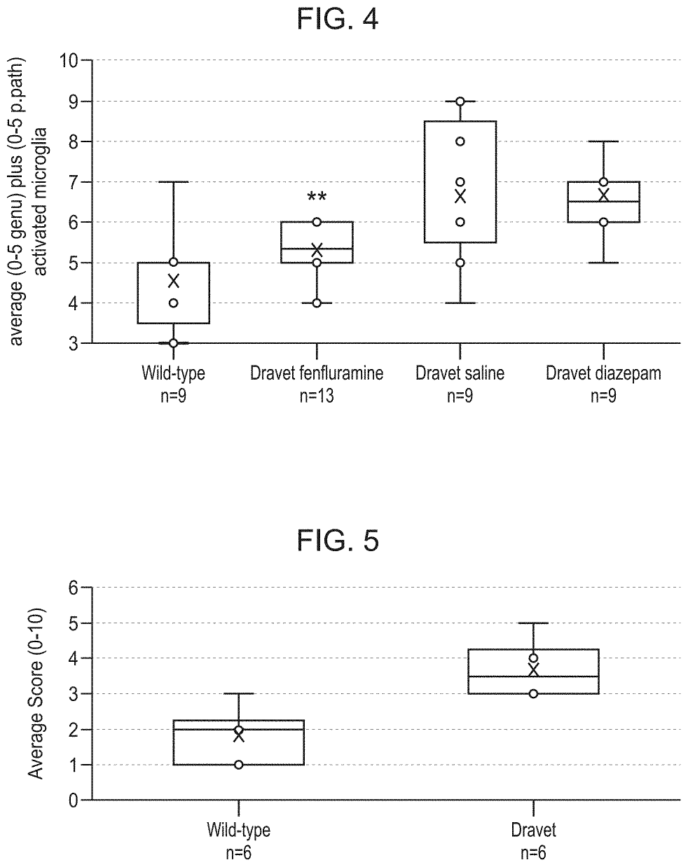

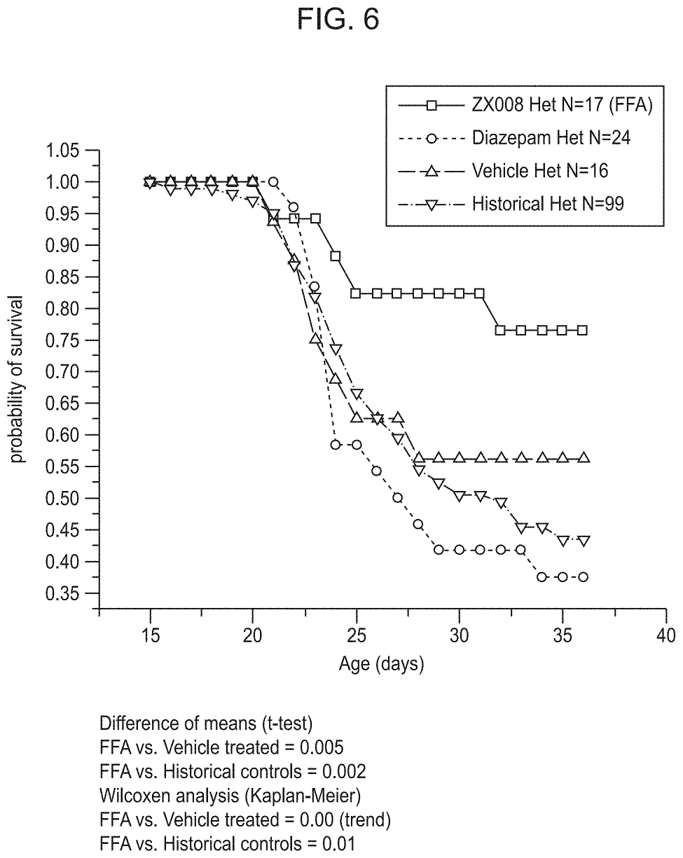

Table 2 shows the average scores from the genu and perforant pathway on a scale of 0-5 as described above (PP=perforant pathway) and as the sum of the average scores from the two brain tissues. Mice with the Scn1a+/− Dravet mutation are indicated as “Het” and animals without the Dravet mutation (Scn1a+/+) are indicated by “WT”. Fenfluramine-treated animals are abbreviated as “FFA”. P-values were calculated using a two-tailed t-test with unequal variances. These data are shown graphically in FIG. 4. These data show that administration of fenfluramine reduces levels of microglia known to respond to myelin pathology and damage within the context of a mouse model of a human disease that is known to have myelin damage.

| Intervention | FFA | FFA | Vehicle | Vehicle | Diazepam | Diazepam | FFA | Vehicle | Diazepam |
|---|---|---|---|---|---|---|---|---|---|
| Het mice | PP | Genu | PP | Genu | PP | Genu | PP + | PP + | PP + |
| | | | | | | | Genu | Genu | Genu |
| CD11b | 2.77 | 2.54 | 3.22 | 3.44 | 3.67 | 3.00 | 5.31 | 6.67 | 6.67 |
| Avg Score | | | | | | | | | |
| stdev | 0.44 | 0.52 | 0.67 | 1.13 | 0.50 | 2.00 | 0.75 | 1.73 | 0.87 |

| | | | |
|---|---|---|---|
| WT mice | PP | Genu | PP + |
| | | | Genu |
| CD11b | 2.56 | 2.00 | 4.56 |
| Avg Score | | | |
| stdev | 0.53 | 1.00 | 1.24 |

CD11b P-values: Combined PP and Genu p = 0.05 Het FFA vs Het vehicle.

### Example 4

### Fenfluramine in an Animal Model of Neonatal Hypoxia

Chronic neonatal hypoxia is a clinically relevant model of premature brain injury caused by insufficient gas exchange from poor lung development. This hypoxic state is a significant contributor to diffuse white matter injury (DWMI), which is common in infants born prematurely. Chronic hypoxia can cause myelination abnormalities. A mouse model of chronic hypoxia has been previously described [Scafidi et al., Nature volume 506, pages 230-234 (2014)]. This model can be used to evaluate the effect of fenfluramine on oligodendrocyte regeneration and remyelination following hypoxia.

Mice are randomly selected to undergo hypoxic rearing or to serve as normoxic controls. Hypoxic mice are placed in a sealed chamber maintaining O2 concentration at 10.5% by displacement with N2 as described previously (Raymond et al., J Neurosci 31:17864-17871, 2011; Bi et al., J Neurosci 31:9205-9221, 2011; Jablonska et al., J Neurosci 32:14775-14793, 2012). Hypoxia is initiated at post-natal day (P)3 and continues for 8 days until P11. This time frame in rodent white matter oligodendrocyte development reproduces changes that occur at 23-40 weeks of gestation in the human brain (Back et al., J Neurosci 21:1302-1312, 2001). Age- and strain-matched mice serve as normoxic controls.

Hypoxic mice and normoxic control mice are randomized to receive daily injections of fenfluramine (1 mg/kg/day) or vehicle. In some examples, administration of fenfluramine (or vehicle) is initiated at P11. In other examples, treatment is initiated at P3 or any time between P3 and P11. Multiple daily doses of fenfluramine (and vehicle) can be administered. Following the desired course of treatment, mice are sacrificed, and brain sections are prepared and processed to evaluate myelin thickness and the number of oligodendrocyte precursor cells in the white matter as described (Scafidi et al., Nature doi: 10.1038/nature12880 [Epub ahead of print], Dec. 25, 2013).

Notwithstanding the appended clauses, the disclosure is also defined by the following clauses:
Clause 1. A method of treatment, comprising:

administering to a subject diagnosed with a demyelination disorder a therapeutically effective amount of fenfluramine or a pharmaceutically acceptable salt thereof.
Clause 2. The method of clause 1, wherein the demyelination disorder is multiple sclerosis (MS).
Clause 3. The method of clause 1, wherein the demyelination disorder is an epileptic encephalopathy.
Clause 4. The method of clause 1, wherein the demyelination disorder is insufficient myelination.
Clause 5. The method of clause 1, wherein the demyelination disorder is underdevelopment of myelin sheaths.
Clause 6. The method of any one of clauses 1-5, wherein the administering is by a route selected from the group consisting of orally, parenterally, and topically.
Clause 7. The method of any one of clauses 1-6, wherein the fenfluramine or pharmaceutically acceptable salt thereof is administered at a dose of about 0.5 mg/kg/day to about 5 mg/kg/day.
Clause 8. The method of any one of clauses 1-7, wherein the fenfluramine or pharmaceutically acceptable salt thereof is administered at a dose of about 0.1 mg/kg/day to about 2 mg/kg/day.
Clause 9. The method of any one of clauses 1-8, wherein the administering is at an interval selected from the group consisting of twice daily, once daily, twice weekly, and once weekly.
Clause 10. The method of any one of clauses 1-9, wherein the subject is diagnosed based on data selected from the group consisting of a magnetic resonance image (MRI), electromyography (EMG), a nerve conductivity study (NCV), an evoked potential study, a lumbar puncture (LP), history of pain, history of nausea, history of vomiting, history of fever, muscle strength, nerve sensation, motor coordination, ability to walk, an eye exam, or a combination thereof.
Clause 11. A method of treatment, comprising:

administering to a patient diagnosed with a condition associated with demyelination a therapeutically effective amount of fenfluramine or a pharmaceutically acceptable salt thereof.
Clause 12. The method of clause 11, wherein the condition associated with demyelination is selected from the group consisting of multiple sclerosis, a leukodystrophy, a leukoencephalopathy, an idiopathic inflammatory demyelinating disease, and Alzheimer's disease.
Clause 13. The method of clause 12, wherein the condition associated with demyelination is multiple sclerosis.
Clause 14. The method of clause 13, wherein the multiple sclerosis is selected from the group consisting of relapsing-remitting multiple sclerosis, primary-progressive multiple sclerosis, secondary-progressive multiple sclerosis, and progressive-relapsing multiple sclerosis.
Clause 15. The method of clause 11, wherein the condition associated with demyelination is selected from the group consisting of central pontine myelinolysis, acute disseminated encephalomyelitis, Balo concentric sclerosis, Marburg multiple sclerosis, tumefactive multiple sclerosis, diffuse myelinoclastic sclerosis, acute hemorrhagic leukoencephalitis, neuromyelitis optica, a chronic inflammatory demyelinating polyneuropathy, Leber hereditary optic neuropathy, multifocal motor neuropathy, paraproteinemic demyelinating polyneuropathy, tropical spastic paraparesis, a Guillain-Barre syndrome, infantile Refsum disease, adult Refsum disease 1, adult Refsum disease 2, Zellweger syndrome, X-linked adrenoleukodystrophy (X-ALD), metachromatic leukodystrophy, Krabbe disease, Pelizaeus-Merzbacher disease, Canavan disease, Alexander disease, Binswanger's disease, peroneal muscular atrophy, cerebrotendinous xanthomatosis, leukoencephalopathy with vanishing white matter, toxic leukoencephalopathy, van der Knaap disease, progressive multifocal leukoencephalopathy, Marchiafava-Bignami disease, and transverse myelitis.
Clause 16. The method of any one of clauses 11-15, wherein the administering is by a route selected from the group consisting of orally, parenterally, and topically.
Clause 17. The method of any one of clauses 11-16, wherein the fenfluramine or pharmaceutically acceptable salt thereof is administered at a dose of about 0.5 mg/kg/day to about 5 mg/kg/day.
Clause 18. The method of any one of clauses 11-17, wherein the fenfluramine or pharmaceutically acceptable salt thereof is administered at a dose of about 0.1 mg/kg/day to about 2 mg/kg/day.
Clause 19. The method of any one of clauses 11-18, wherein the administering is at an interval selected from the group consisting of twice daily, once daily, twice weekly, and once weekly.
Clause 20. The method of any one of clauses 11-19, further comprising diagnosing the subject with the condition associated with demyelination.
Clause 21. The method of any one of claims 11-20, wherein the subject is diagnosed based on data selected from the group consisting of a magnetic resonance image (MRI), electromyography (EMG), a nerve conductivity study (NCV), an evoked potential study, a lumbar puncture (LP), history of pain, history of nausea, history of vomiting, history of fever, muscle strength, nerve sensation, motor coordination, ability to walk, an eye exam, or a combination thereof.
Clause 22. A method of treatment, comprising:

administering to the patient diagnosed with a symptom of a disease a therapeutically effective amount of fenfluramine or a pharmaceutically acceptable salt thereof,

wherein the symptom of the disease is selected from the group consisting of a lack of sphincter control, erectile dysfunction, paraparesis, ataxia, adrenocortical insufficiency, progressive neuropathy, paresthesia, dysarthria, dysphagia, clonus, or any combination thereof.
Clause 23. The method of clause 22, wherein the administering is by a route selected from the group consisting of orally, parenterally, and topically.
Clause 24. The method of any of clauses 22-23, wherein the fenfluramine or pharmaceutically acceptable salt thereof is administered at a dose of about 0.5 mg/kg/day to about 5 mg/kg/day.
Clause 25. The method of any of clauses 22-24, wherein the fenfluramine or pharmaceutically acceptable salt thereof is administered at a dose of about 0.1 mg/kg/day to about 2 mg/kg/day.
Clause 26. The method of any of clauses 22-25, wherein the administering is at an interval selected from the group consisting of twice daily, once daily, twice weekly, and once weekly.
Clause 27. The method of any one of clauses 22-26, further comprising diagnosing the subject with the symptom of the disease.

All publications, patents, and patent applications mentioned in the above specification are hereby incorporated by reference. Applicants make no admission that any such references, patents and patent publications cited herein constitute prior art. Various modifications and variations of the described device and methods of use of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

1. A method of treatment, comprising:
administering to a subject diagnosed with a demyelination disorder a therapeutically effective amount of fenfluramine or a pharmaceutically acceptable salt thereof.

2. The method of claim 1, wherein the demyelination disorder is multiple sclerosis (MS).

3. The method of claim 1, wherein the demyelination disorder is an epileptic encephalopathy.

4. The method of claim 1, wherein the demyelination disorder is insufficient myelination.

5. The method of claim 1, wherein the demyelination disorder is underdevelopment of myelin sheaths.

6. The method of claim 1, wherein the administering is by a route selected from the group consisting of orally, parenterally, and topically.

7. The method of claim 1, wherein the fenfluramine or pharmaceutically acceptable salt thereof is administered at a dose of about 0.5 mg/kg/day to about 5 mg/kg/day.

8. The method of claim 1, wherein the fenfluramine or pharmaceutically acceptable salt thereof is administered at a dose of about 0.1 mg/kg/day to about 2 mg/kg/day.

9. The method of claim 1, wherein the administering is at an interval selected from the group consisting of twice daily, once daily, twice weekly, and once weekly.

10. The method of claim 1, wherein the subject is diagnosed based on data selected from the group consisting of a magnetic resonance image (MRI), electromyography (EMG), a nerve conductivity study (NCV), an evoked potential study, a lumbar puncture (LP), history of pain, history of nausea, history of vomiting, history of fever, muscle strength, nerve sensation, motor coordination, ability to walk, an eye exam, or a combination thereof.

11. A method of treatment, comprising:
administering to a patient diagnosed with a condition associated with demyelination a therapeutically effective amount of fenfluramine or a pharmaceutically acceptable salt thereof.

12. The method of claim 11, wherein the condition associated with demyelination is selected from the group consisting of multiple sclerosis, a leukodystrophy, a leukoencephalopathy, an idiopathic inflammatory demyelinating disease, and Alzheimer's disease.

13. The method of claim 12, wherein the condition associated with demyelination is multiple sclerosis.

14. The method of claim 13, wherein the multiple sclerosis is selected from the group consisting of relapsing-remitting multiple sclerosis, primary-progressive multiple sclerosis, secondary-progressive multiple sclerosis, and progressive-relapsing multiple sclerosis.

15. The method of claim 11, wherein the condition associated with demyelination is selected from the group consisting of central pontine myelinolysis, acute disseminated encephalomyelitis, Balo concentric sclerosis, Marburg multiple sclerosis, tumefactive multiple sclerosis, diffuse myelinoclastic sclerosis, acute hemorrhagic leukoencephalitis, neuromyelitis optica, a chronic inflammatory demyelinating polyneuropathy, Leber hereditary optic neuropathy, multifocal motor neuropathy, paraproteinemic demyelinating polyneuropathy, tropical spastic paraparesis, a Guillain-Barre syndrome, infantile Refsum disease, adult Refsum disease 1, adult Refsum disease 2, Zellweger syndrome, X-linked adrenoleukodystrophy (X-ALD), metachromatic leukodystrophy, Krabbe disease, Pelizaeus-Merzbacher disease, Canavan disease, Alexander disease, Binswanger's disease, peroneal muscular atrophy, cerebrotendinous xanthomatosis, leukoencephalopathy with vanishing white matter, toxic leukoencephalopathy, van der Knaap disease, progressive multifocal leukoencephalopathy, Marchiafava-Bignami disease, and transverse myelitis.

16. The method of claim 11, wherein the fenfluramine or pharmaceutically acceptable salt thereof is administered at a dose of about 0.5 mg/kg/day to about 5 mg/kg/day.

17. The method of claim 11, wherein the fenfluramine or pharmaceutically acceptable salt thereof is administered at a dose of about 0.1 mg/kg/day to about 2 mg/kg/day.

18. The method of claim 11, further comprising diagnosing the subject with the condition associated with demyelination.

19. The method of claim 11, wherein the subject is diagnosed based on data selected from the group consisting of a magnetic resonance image (MRI), electromyography (EMG), a nerve conductivity study (NCV), an evoked potential study, a lumbar puncture (LP), history of pain, history of nausea, history of vomiting, history of fever, muscle strength, nerve sensation, motor coordination, ability to walk, an eye exam, or a combination thereof.

20. A method of treatment, comprising:
administering to the patient diagnosed with a symptom of a disease a therapeutically effective amount of fenfluramine or a pharmaceutically acceptable salt thereof,
wherein the symptom of the disease is selected from the group consisting of a lack of sphincter control, erectile dysfunction, paraparesis, ataxia, adrenocortical insufficiency, progressive neuropathy, paresthesia, dysarthria, dysphagia, clonus, or any combination thereof.
